(19) United States
(12) Patent Application Publication
(10) Pub. No.: US 2023/0355374 A1
(43) Pub. Date: Nov. 9, 2023

(54) TENDON REPAIR ANCHOR

(71) Applicant: Quadvantage Technology, Inc., Raleigh, NC (US)

(72) Inventors: Paul Leach Burroughs, III, Raleigh, NC (US); Thomas Gutierrez, Macon, NC (US)

(21) Appl. No.: 18/143,663

(22) Filed: May 5, 2023

Related U.S. Application Data
(60) Provisional application No. 63/386,042, filed on Dec. 5, 2022.
(60) Provisional application No. 63/364,202, filed on May 5, 2022.

(51) Int. Cl. A61F 2/08

(52) U.S. Cl. CPC A61F 2/0811; A61F 2230/0067; A61F 2002/0081

(57) ABSTRACT

The present disclosure provides a tendon repair anchor. An anchor plug is inserted into an anchor receptacle seated in a bone of a graft recipient. Sloped protrusions on the anchor plug interact with protrusions in the anchor receptacle to secure the anchor plug within the anchor receptacle and generally allow movement in a direction that more tightly secures the tendon graft to the bone. By allowing such unidirectional movement, slack in the sutures that secure the tendon graft to the anchor plug will not prevent the tendon graft from being properly positioned relative to the bone. Proper placement of the tendon graft allows for better healing and improves the likelihood of proper tendon replacement and tensioning.

### PRIORITY APPLICATIONS

The present application claims priority to U.S. Provisional Patent Application Ser. No. 63/364,202 filed on May 5, 2022, and entitled, “TENDON REPAIR ANCHOR,” the contents of which is incorporated herein by reference in its entirety.

The present application also claims priority to U.S. Provisional Patent Application Ser. No. 63/386,042 filed on Dec. 5, 2022, and entitled, “TENDON REPAIR ANCHOR,” the contents of which is incorporated herein by reference in its entirety.

### FIELD OF THE DISCLOSURE

The present disclosure relates to a device that assists in attaching a tendon to bone such as during anterior cruciate ligament (ACL) replacement.

### BACKGROUND

Most people can go through the majority of their life without ever caring or knowing how complicated a structure the knee that helps them walk is. However, the knee remains a fragile mechanical structure that is readily susceptible to damage. While medical advances have made repairing the knee possible, repair of certain types of injuries results in other long-term effects. To assist the reader in appreciating the elegance of the present disclosure, FIG. 1 is provided with a brief explanation of the components of the knee.

For the purposes of the present disclosure, and as illustrated, the knee may be composed of the quadriceps muscles 100, the femur 102, the articular cartilage 104, the lateral condyle 106, the posterior cruciate ligament 108, the anterior cruciate ligament (ACL) 110, the lateral collateral ligament 112, the fibula 114, the tibia 116, the patellar tendon 118, the meniscus 120, the medial collateral ligament 122, the patella 124 (shown slightly displaced to the side—it normally rests in the center of the knee), and the quadriceps tendon 126.

ACL tears are common in athletes and are usually season-ending injuries. The ACL 110 cannot heal—it must be surgically reconstructed. The reconstruction requires replacement tissue. The most common tissue used is a central slip of the patient's own patellar tendon 118. In practice, the patellar tendon 118 has proven to be generally effective, but the size of the graft that can be used is limited to the size of the patient's own patellar tendon 118. As a rule of thumb, only a third of the patellar tendon 118 may be harvested as a graft. Thus, a doctor will measure the width of the patellar tendon 118, divide by three, and take the middle third of the patellar tendon 118. Such harvested grafts are rarely more than ten millimeters (10 mm) wide and may be smaller. Taking this tissue from a person's patellar tendon 118 also causes significant pain and discomfort in the post-operative healing period, which may last up to a year, and up to twenty (20) percent of these patients are left with chronic anterior knee pain.

Some doctors recommend and use other graft sources, such as cadaver grafts, but cadaver grafts have a higher failure rate. Additionally, there is a non-zero chance of disease transmission or rejection by the patient's immune system. As a final drawback, cadaver grafts are usually quite expensive and may not be covered by some insurance companies.

Other doctors use hamstring tendons (e.g., the distal semitendinosus tendon) because the scar created during harvesting is relatively small and there is less pain during the rehabilitation, but again, the hamstring tendon has its own collection of disadvantages. The disadvantages include the fact that once the graft is taken, a patient's hamstring will never recover to its previous strength. Further, all hamstring reconstructions stretch and are looser than the original ACL 110. This loosening is particularly problematic in younger female athletes.

Another alternative graft source is the quadriceps tendon 126. The quadriceps tendon 126 is larger and stronger than either the patellar tendon 118 or the hamstring tendon. The quadriceps tendon 126 is likewise stiffer and less prone to stretching or plastic deformation. However, the qualities that make the quadriceps tendon 126 attractive also contribute to the difficulty in harvesting a graft from the quadriceps tendon 126. Existing surgical implements require a large incision up the longitudinal axis of the femur 102 on the front or ventral/anterior side of the thigh to cut down to the level of the quadriceps tendon 126, resulting in a large post-operative scar. Additionally, the quadriceps tendon 126 has a consistency similar to the proverbial shoe leather, making it difficult to cut. However, an ACL 110 repaired with grafts from the quadriceps tendon 126 generally results in almost no anterior knee pain postoperatively over the short or long term and patients recover quicker.

Regardless of what tendon is harvested, there remains a challenge in attaching the tendon to the bone of the graft recipient, particularly when no bone plug is taken with the graft at the time of harvesting. Accordingly, there is room for innovation in this space.

### SUMMARY

The present disclosure provides a tendon repair anchor. Exemplary aspects of the present disclosure relate to an anchor plug to which a tendon graft may be secured. The anchor plug is inserted into an anchor receptacle seated in a bone of a graft recipient. Sloped protrusions on the anchor plug interact with protrusions in the anchor receptacle to secure the anchor plug within the anchor receptacle and generally allow movement in a direction that more tightly secures the tendon graft to the bone. By allowing such unidirectional movement, slack in the sutures that secure the tendon graft to the anchor plug will not prevent the tendon graft from being properly positioned relative to the bone. Proper placement of the tendon graft allows for better healing and improves the likelihood of proper tendon replacement and tensioning. While a primary focus is on use in repairing an anterior cruciate ligament (ACL), the present disclosure has other tendon repair applications and is not limited to ACL repair.

In this regard, in one aspect, a fixation assembly is disclosed. The fixation assembly comprises an anchor plug comprising an elongated body comprising a plurality of tapering conical sections. The fixation assembly also comprises an anchor receptacle comprising an interior passage with interior teeth configured to interengage with the plurality of tapering conical sections of the anchor plug when the anchor plug is inserted into the interior passage.

Those skilled in the art will appreciate the scope of the disclosure and realize additional aspects thereof after reading the following detailed description in association with the accompanying drawings.

### BRIEF DESCRIPTION OF THE DRAWINGS

The accompanying drawings incorporated in and forming a part of this specification illustrate several aspects of the disclosure, and together with the description serve to explain the principles of the disclosure.

FIG. 1 illustrates a conventional knee;

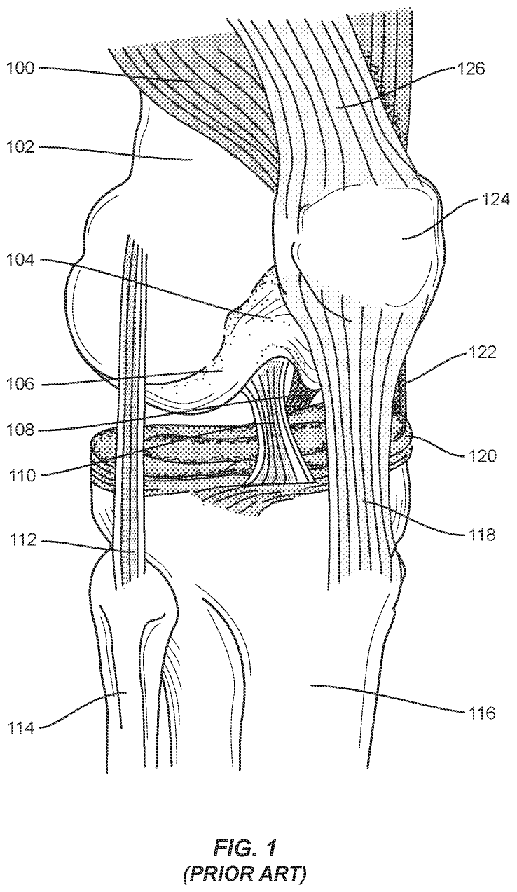

FIG. 2 shows an endobutton being used with a graft for tendon repair;

FIG. 3A-3D are various views of an anchor plug for use in securing a tendon graft to a bone according to an exemplary aspect of the present disclosure;

FIGS. 4A-4D are various view of an anchor receptacle that works with the anchor plug of FIGS. 3A-3D according to an exemplary aspect of the present disclosure;

FIG. 4E is a cross-sectional view of the anchor receptacle taken along lines 4E of FIG. 4C;

FIG. 5 is a cross-sectional elevational view of the anchor plug inserted into the anchor receptacle;

FIG. 6 is a perspective view of a tendon graft attached to the anchor plug being pulled into an anchor receptacle;

FIG. 7 is a view of an anchor receptacle inserted into a bone condyle showing how the anchor works relative to the bone;

FIG. 8 is a perspective view of an alternate anchor receptacle with a modified compression sleeve;

FIG. 9 is a perspective view of an alternate anchor receptacle that is designed to work with a washer to assist in placement against a bone;

FIG. 10 is a side view of an alternate threading technique that uses an alternate anchor receptacle;

FIGS. 11-14 show various ways to attach a graft or workpiece to an anchor plug; and

FIG. 15 is a flowchart of a process for attaching a receptacle anchor to a bone such as a femur.

### DETAILED DESCRIPTION

The embodiments set forth below represent the necessary information to enable those skilled in the art to practice the disclosure and illustrate the best mode of practicing the disclosure. Upon reading the following description in light of the accompanying drawings, those skilled in the art will understand the concepts of the disclosure and will recognize applications of these concepts not particularly addressed herein. It should be understood that these concepts and applications not particularly addressed fall within the scope of the disclosure and the only limitations are found in the accompanying claims.

The present disclosure provides a tendon repair anchor. Exemplary aspects of the present disclosure relate to an anchor plug to which a tendon graft may be secured. The anchor plug is inserted into an anchor receptacle seated in a bone of a graft recipient. Sloped protrusions on the anchor plug interact with protrusions in the anchor receptacle to secure the anchor plug within the anchor receptacle and generally allow movement in a direction that more tightly secures the tendon graft to the bone. By allowing such unidirectional movement, slack in the sutures that secure the tendon graft to the anchor plug will not prevent the tendon graft from being properly positioned relative to the bone. Proper placement of the tendon graft allows for better healing and improves the likelihood of proper tendon replacement and tensioning. While a primary focus is on use in repairing an anterior cruciate ligament (ACL), the present disclosure has other tendon repair applications and is not limited to ACL repair.

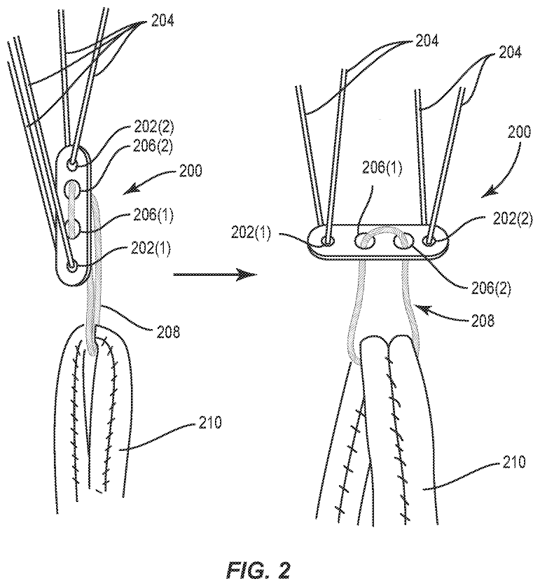

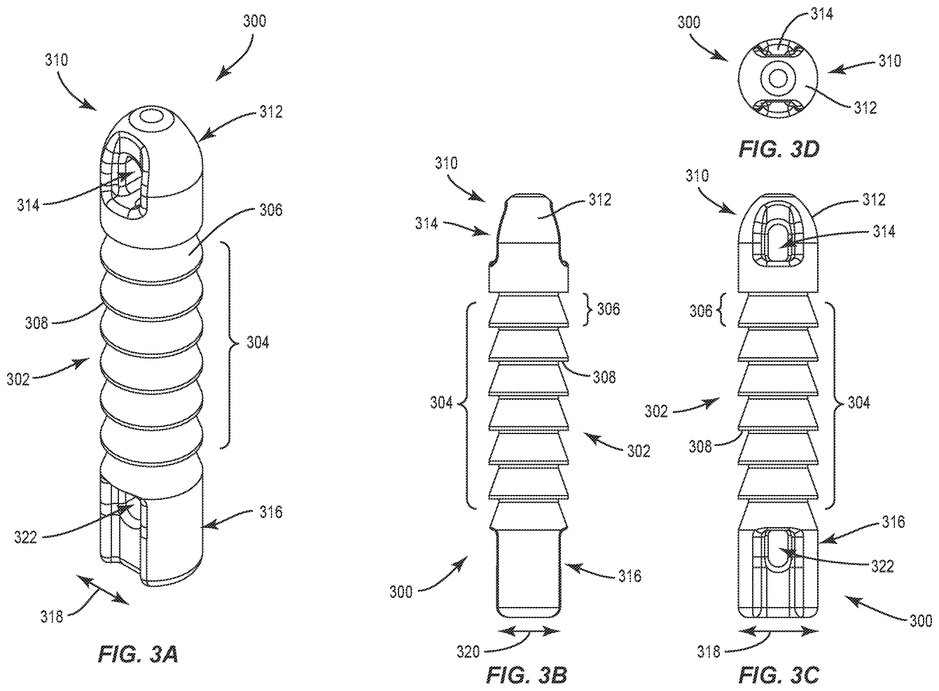

Before addressing exemplary aspects of the present disclosure, it is worth noting what is currently being done and the shortcomings associated with the conventional approach as shown in FIG. 2. A discussion of exemplary aspects of the present disclosure begins below with reference to FIG. 3.

Traditionally, a graft is harvested from a donor source (whether it be from the same person (e.g., a patella tendon, a quadriceps tendon, or the like being used to repair an ACL (or other tendon)) or from a cadaver). In many instances, the graft does not have a bone spur or bone plug that can be used to facilitate attachment at the repair site. The common approach in this case is the use of an endobutton 200 illustrated in FIG. 2. The endobutton 200 has a flat portion with two end apertures 202(1), 202(2) that are designed to have sutures or similar thread-like material 204 passing therethrough. Two interior apertures 206(1)-206(2) accommodate a graft loop 208. A graft 210 is passed through the graft loop 208. However, the size of the graft loop 208 is not adjustable. Whether the graft loop 208 is preformed or tied, the graft loop 208 is not adjustable once tied and cannot be adjusted when inside the knee. Accordingly, there may be slack in the graft loop 208 that makes the graft 210 loose therewithin. In contrast, if the graft loop 208 is too small or tied too tightly, the endobutton 200 will not be able to be flipped. That is, the graft loop 208 may be too short or too long and the end result is that the graft 210 will be too deep or too shallow in the femoral tunnel. While the endobutton 200 may be securely affixed to the bone at the repair site, the tension on the graft cannot be adjusted, and thus the fixed excessive slack (or excessive tightness) in the graft loop 208 may compromise the position of the graft 210 in the femoral tunnel. Note further that while there are “adjustable” fixation devices sometimes used with longer “looped” hamstring grafts, these adjustable devices will not work with a graft made from a quadriceps tendon as quadricep tendon grafts are shorter, even with a bone plug, and cannot use those adjustable implants.

Accordingly, there is room for a better way to attach a graft at a repair site. Again, while the discussion focuses on ACL repair, other tendon repair may also benefit from aspects of the present disclosure. Exemplary aspects of the present disclosure provide an anchor that allows the surgeon to determine exactly how deep or how shallow they wish the graft to be positioned in the tunnel. Further, aspects of the present disclosure provide an adjustable option for shorter, non-looped grafts such as a quadriceps tendon graft.

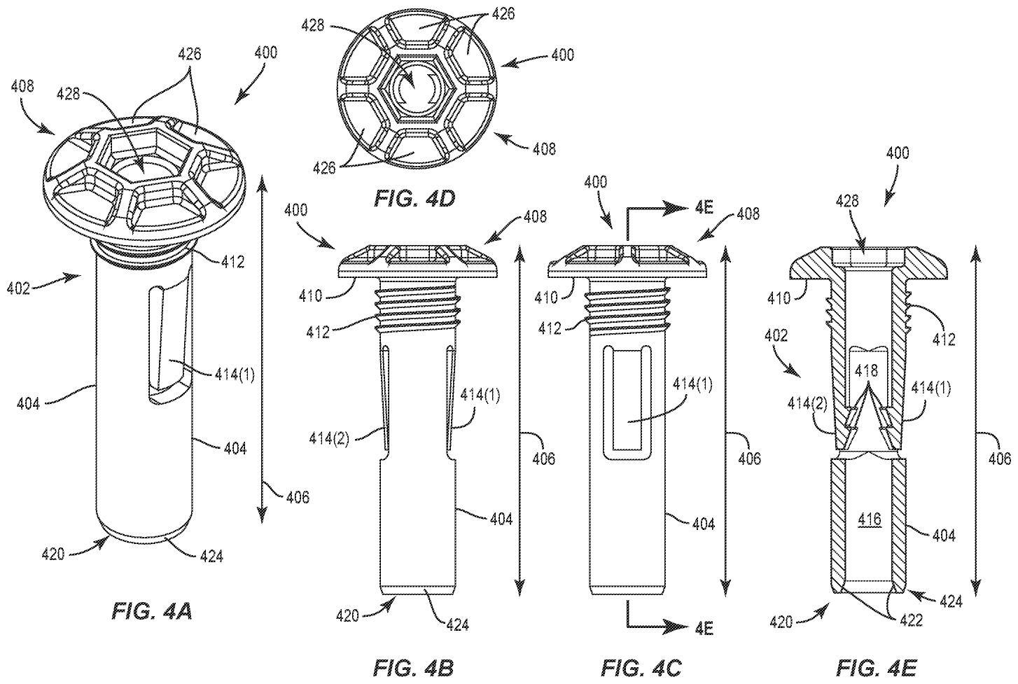

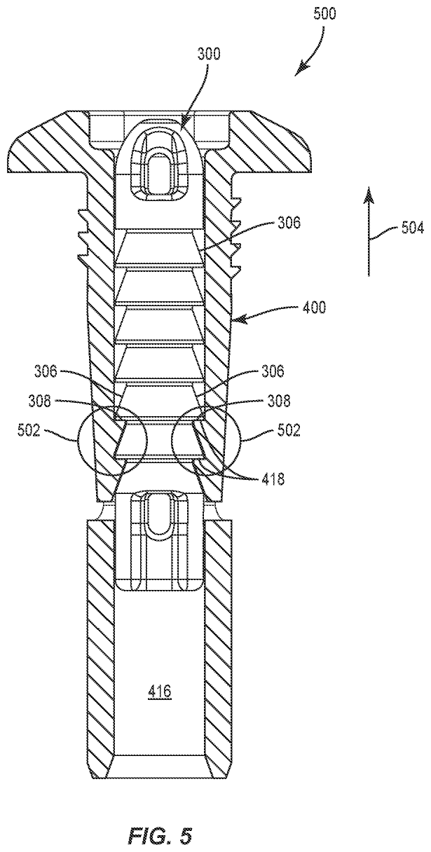

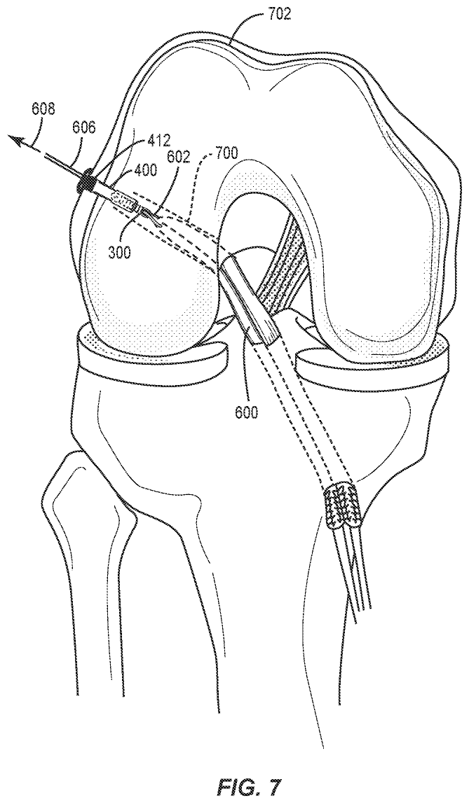

In this regard, FIGS. 3A-3D illustrate an anchor stud or plug 300, which has an elongate body 302 with a middle section 304 that has a plurality of tapering conical sections 306 each having a shoulder or lip 308. An insertion end 310 may be smoothly tapered or have an arcuate conical tip 312 that delimits an aperture 314 that extends through the conical tip 312. In practice, as better seen in FIG. 6, a suture thread may be threaded through the aperture 314 to assist in pulling the anchor plug 300 into an anchor receptacle 400 (FIG. 4A). While the aperture 314 may take any number of shapes, in an exemplary aspect, the aperture 314 includes smooth rounded surfaces and an absence of hard corners to avoid cutting a suture thread placed therethrough.

The anchor plug 300 may further include a distal end 316 that has a general diameter 318 less than or equal to a diameter of the lips 308. The distal end 316 may have a lateral dimension 320 less than the general diameter 318, and which is also less than the diameter of the lips 308. The distal end 316 may further delimit a distal aperture 322 through which a suture thread may be inserted as better seen in FIG. 6. Again, the edges of the distal aperture 322 may be rounded or smooth to avoid cutting a suture thread placed therethrough. In an exemplary aspect, the distal aperture 322 may have a diameter of approximately 4.25 millimeters (mm) if circular, but other shapes such as an oval or ellipse may also be used.

In an exemplary aspect, the anchor plug 300 may be a carbon fiber or polymeric material and may be between about 4 and 8 mm long. In a further exemplary aspect, the lips 308 may be slightly flexible or resilient to assist in passing into and through the anchor receptacle 400 while still maintaining enough rigidity to avoid backward motion as better explained below. Alternatively, the flexibility may be built into the anchor receptacle 400 and the anchor plug 300 may be substantially rigid.

In another exemplary aspect, the distal aperture 322 may be sized to accommodate an actual tendon graft rather than merely a suture thread. In such an instance, the tendon graft might be looped through the distal aperture 322 and secured to itself using sutures or the like. In general, a cross-sectional area of about 113 mm² should fit any size graft. That is, the largest likely graft would have a lateral dimension of 12 mm or a radius of 6 mm, and given that area equals πr², a cross-sectional area of 113 mm² would allow such a 12 mm graft to be placed through the distal aperture 322. While specific values are provided by way of example, it should be appreciated that other dimensions may also be used without departing from the present disclosure.

FIGS. 4A-4E illustrate the anchor receptacle 400, with FIG. 4E being a cross-sectional view taken along lines 4E-4E of FIG. 4C. The anchor receptacle 400 includes an elongate body 402 having a generally smooth exterior shaft 404 along a longitudinal axis 406. The anchor receptacle 400 further includes a head portion 408 having a lateral dimension greater than the lateral dimension of the shaft 404 such that a shoulder or flange 410 is defined on a lower surface of the head portion 408.

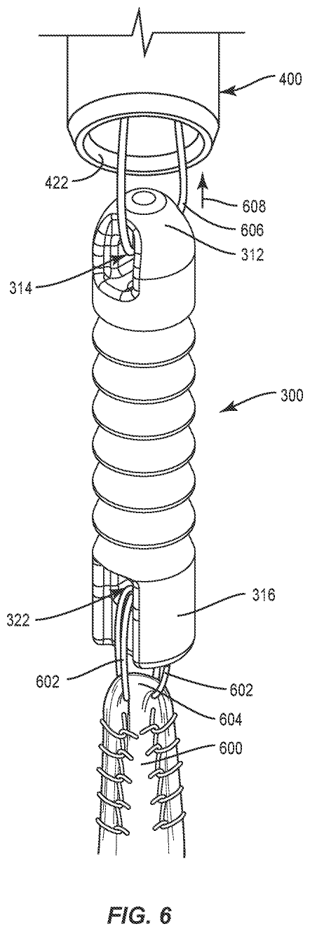

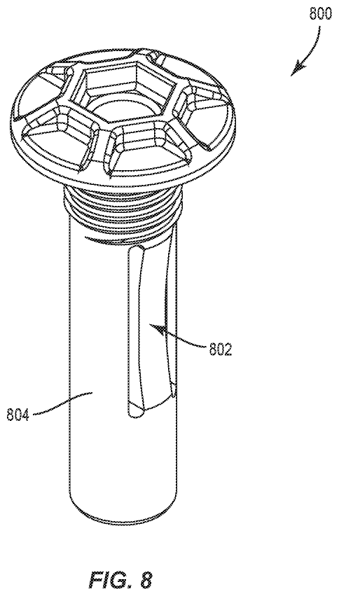

The shaft 404 may include threads 412 proximate the head portion 408. The threads 412 may extend a partial length or possibly the entire length of the anchor receptacle 400. Additionally, the shaft 404 may include inwardly tapered clips 414(1)-414(2) positioned on opposite sides of the shaft 404. As better seen in FIG. 4E, the shaft 404 may include an interior passage 416 that extends the length of the longitudinal axis 406 of the elongate body 402. The clips 414(1)-414(2) may be resilient to allow the anchor plug 300 to pass therebetween. For example, the resilience may be provided by the open lower end of the clips 414(1)-414(2), a reduced thickness of the clips 414(1)-414(2) relative to the rest of the body, a material change, or the like. The clips 414(1)-414(2) may include interior teeth 418, with upwardly facing flanges or shoulders within the interior passage 416. The interior passage 416 is sized so as to fit the anchor plug 300 therewithin as better seen in FIG. 5, discussed below. A distal end 420 of the interior passage 416 may include an interior taper 422 to assist in insertion of the anchor plug 300. Additionally, the distal end 420 may include an exterior taper 424 to assist in insertion of the anchor receptacle 400 into a bone as better seen in FIG. 7, discussed below.

The head portion 408 may include cutouts 426 that may be used to twist the anchor receptacle 400 manually, such as by using fingers. Additionally, the interior passage 416 may adopt a hex-shaped configuration 428 so that a cannulated hex driver (e.g., an allen wrench) or cannulated screwdriver may be used to drive the anchor receptacle 400 into a bone as better seen in FIG. 7, discussed below. Note also that the cutouts 426 may be configured to interoperate with such a cannulated screwdriver if desired. In an exemplary aspect, the hex-shaped configuration 428 has a greater lateral dimension than the main portion of the interior passage 416. Note also that the cannulated hex driver and/or cannulated screwdriver may be shorter than normal (e.g., half the normal length) and may have graduations or indicia on a side that may be used to confirm a depth of insertion.

The mating of the anchor plug 300 and the anchor receptacle 400 is seen in fixation assembly 500 in FIG. 5. In particular, the anchor plug 300 slides within the interior passage 416. The lips 308 of the tapering conical sections 306 interengage with the interior teeth 418 (see generally areas 502). The clips 414(1)-414(2) expand as the tapering conical sections 306 are drawn upwardly through the interior passage 416, and then ratchet down as the lip 308 passes such that the movement of the anchor plug 300 is in a single direction 504 along the longitudinal axis 406. The lips 308 circumnavigate the anchor plug 300, so the rotational orientation of the anchor plug 300 relative to the anchor receptacle 400 should not matter. Note that there may be situations where the anchor plug 300 slides through the interior passage 416 such that the conical tip 312 extends pasts the flange 410 while the lips 308 and the interior teeth 418 are still interengaged.

While not illustrated, the present disclosure also contemplates angling the lips 308 and the interior teeth 418 in a complementary fashion akin to rifling or threads such that rotation of the anchor receptacle 400 (e.g., such as by a cannulated screwdriver) may draw the anchor plug 300 up into the anchor receptacle 400.

FIG. 6 provides a perspective view of the anchor plug 300 attached to a graft 600 by a suture thread 602 that is woven into the graft 600 through, for example, a Krakow weave. The suture thread 602 passes through the distal aperture 322 and is tied off. In an exemplary aspect, the suture thread 602 is tied off such that the top 604 of the graft 600 is flush against the distal end 316 of the anchor plug 300. Aspects of the present disclosure are able to correct situations where the suture thread 602 is tied such that there is slack between the graft 600 and the distal end 316 of the anchor plug 300.

At the conical tip 312, a second suture thread 606 may pass through the aperture 314 and the loose ends threaded into the anchor receptacle 400 through the interior passage 416. The ends of the second suture thread 606 are pulled upwardly (e.g., arrow 608 drawing the anchor plug 300 into the interior passage 416 assisted by the conical nature of the conical tip 312 and the interior taper 422. The aperture 314 may be improved by adding a metallic (or other material) insert or eyelet, thus increasing the strength of the aperture 314 and providing a possibly superior surface through spreading the load more evenly and avoiding rough or sharp edges that could damage the suture.

An in-situ view of this process is shown in FIG. 7. A hole or passage 700 is drilled through a condyle on the femur 702 (assuming an ACL repair; if other repair is being done, another bone may be used). The anchor receptacle 400 is then screwed into the passage 700, where the threads 412 may assist in keeping the anchor receptacle 400 secured within the passage 700 and the flange 410 (not seen on FIG. 7) lies flush against the femur 702, preventing over insertion of the anchor receptacle 400. The graft 600 is secured to the anchor plug 300 using the suture thread 602, and the second suture thread 606 is drawn through the passage 700, through the anchor receptacle 400 and out the other side. The surgeon pulls on the second suture thread 606 upwardly as shown by arrow 608. Note that the suture threads 602, 606 are pulled by a guidewire through the anchor receptacle 400 and out of the skin through the cannulated screwdriver, which is left in place to provide a counterforce against the anchor receptacle 400 when pulling the anchor plug 300 through it (in case the threads 412 aren't providing enough force). Note that upwardly in this context is not an absolute term, but is intended to cover pulling away from the femur 702 such that the anchor plug 300 enters the anchor receptacle 400, drawing the graft 600 into the passage 700 such that the femur 702 may heal around the graft 600. The ability to position the anchor plug 300 selectively in the anchor receptacle 400 through the selective use of the interior teeth 418 and the tapering conical sections 306 effectively absorbs any slack in the suture thread 602 such that the graft 600 is still in a desired position relative to the femur 702 for healing. In an exemplary aspect, this passage 700 may be between 10 to 50 mm long. The anchor receptacle 400 may extend approximately 15-25 mm into the passage 700. The strength of the head portion 408 of the anchor receptacle 400 along with the mating of the interior lip 308 with the interior teeth, 418 should be designed to withstand approximately 1000 Newtons of force without deformation.

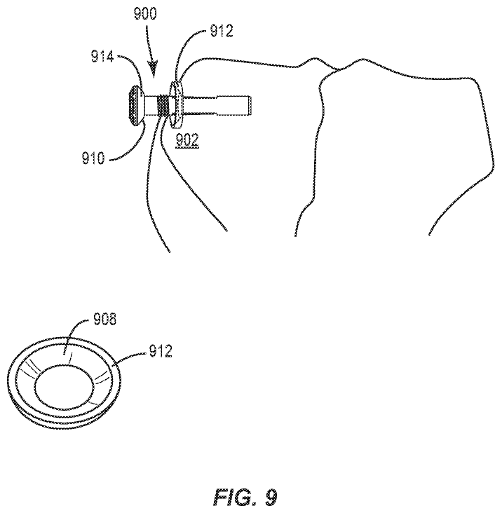

FIG. 8 illustrates an alternate anchor receptacle 800 having a clip 802 (another being present on the opposite side (not shown)) that is continuous with the body 804 rather than open at one end like the clips 414(1)-414(2).

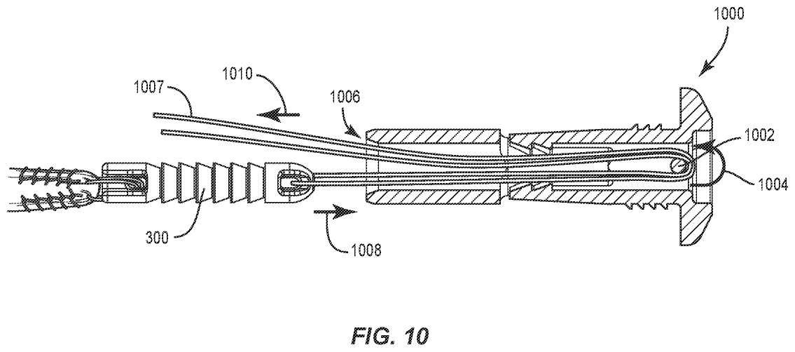

As another possible alternate aspect, an anchor receptacle 900, illustrated in FIG. 9, may include a rounded flange 910 designed to mate with a washer 912. The washer 912 may help prevent edge loading where the anchor receptacle 900 cannot or is not placed exactly 90 degrees to the bone surface 902. The washer 912 may include an internal bevel 908 that mates readily with the slope 914 of the rounded flange 910.

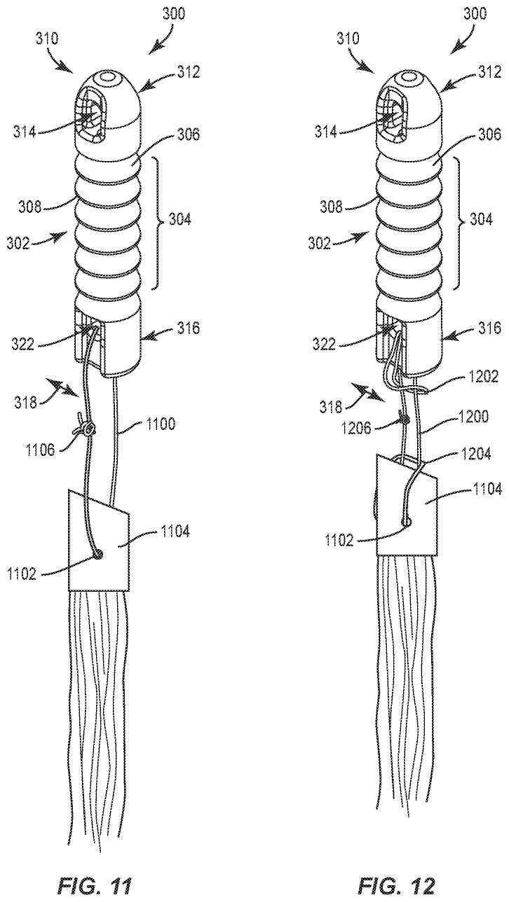

As still another possible aspect, an anchor receptacle 1000, illustrated in FIG. 10, may include a top crossbar 1002 that bisects the top aperture 1004 of the interior passage 1006. A suture thread 1007 may be threaded up (generally noted by 1008) through the interior passage 1006, over the top crossbar 1002 and then back down (generally noted by 1010) through the interior passage 1006. This approach may make it easier to pull the anchor plug 300 into the interior passage 1006 in some instances.

Note also that the anchor receptacle (of any of the types disclosed above) may come in different sizes and a commercial package may include a single anchor stud with multiple differently-sized anchor receptacles (or receptacles of different types (e.g., one receptacle 400, one receptacle 800, and one receptacle 900)).

While early prototyping and testing has proven favorable, as with many prototype devices, room for improvement was found. For example, when the anchor receptacle 400 is inserted into the femoral bone tunnel, the above description notes that a cannulated screwdriver may be used. The anchor receptacle 400 and screwdriver may go over the guidewire into the femur. The guidewire likely has a smaller diameter than the passage 700. Accordingly, it may be advantageous to keep the guidewire centered in the passage 700 so that the edges of the anchor receptacle 400 will line up with the edges of the passage 700.

One solution may be to leave the drill or reamer in the passage 700 while the second suture thread 606 is being passed through the anchor receptacle 400. Once the anchor receptacle 400 starts down the passage 700, the drill can be removed.

Another solution is to add an extension on a tip of the cannulated screwdriver, where the tip extends through the anchor receptacle 400 and out the distal end 420. This may reduce the need to juggle the drill while inserting the anchor receptacle 400.

Additionally, testing has shown that the tighter the anchor plug 300 fits in the anchor receptacle 400, the less the chance of failure. However, the anchor receptacle 400 may not have a tight hold (even with the threads 412). Accordingly, it may be appropriate to leave the cannulated screwdriver engaged with the anchor receptacle 400, pushing the anchor receptacle 400, and particularly the flange 410, against the femur, while at the same time, pulling the second suture thread 606 out a hole in the screwdriver. The force on the second suture thread 606 pulls the anchor plug 300 into the anchor receptacle 400. Too much force may pull the anchor plug 300 too far into the anchor receptacle 400. To prevent this over insertion, the screwdriver may be equipped with a crank or ratchet that would put force on the second suture thread 606, but only in small increments. Another option would be to take a needle driver or hemostat and wrap the second suture thread 606 around same. As the needle driver or hemostat is rotated, the second suture thread 606 is pulled through the anchor receptacle 400.

Exemplary aspects of the present disclosure allow the anchor plug 300 to be positioned adjustably within the anchor receptacle 400. As previously indicated, the graft tissue may be securely fixed or connected to the anchor plug 300 so that the graft tissue is readily drawn up into the passage 700. There are three commonly-used graft materials, any of which may be attached to the anchor plug 300. The three commonly-used graft materials are 1) a bone plug (e.g., a piece of patella attached to a patellar tendon), 2) a looped tendon (e.g., such as a hamstring tendon), or 3) a free end of a tendon (e.g., such as a tendon only quadriceps tendon). Different suture constructs may be utilized depending on the structure of the graft material. These are detailed below.

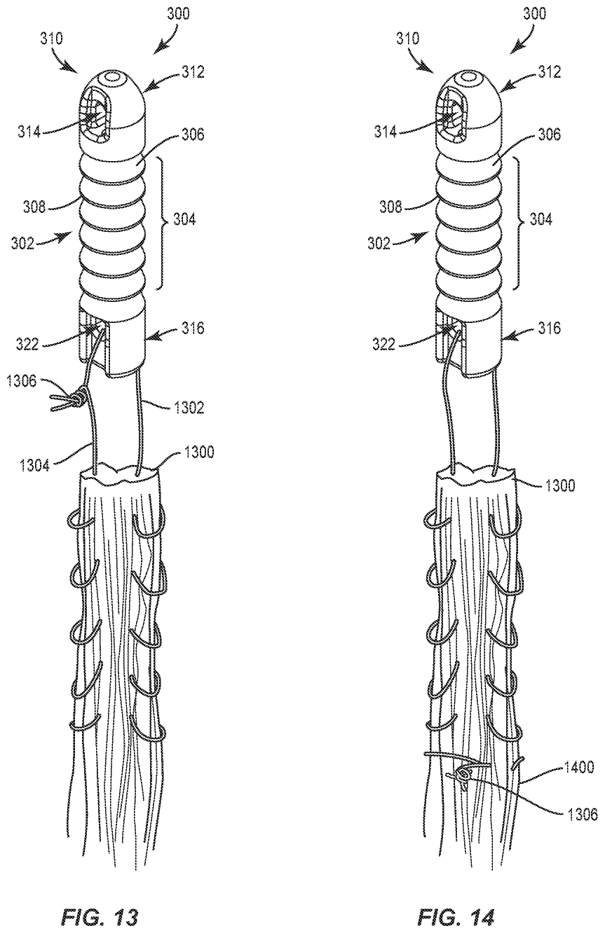

In a first exemplary aspect, where there is bone plug fixation, a #2 or a #5 suture 1100 (single or double stranded) is passed through a drill hole 1102 in a bone plug 1104 and the distal aperture 322 of the anchor plug 300 as shown in FIG. 11. A knot 1106 is hand tied.

In a second exemplary aspect where there is bone plug fixation, a luggage tag suture structure may be formed, as illustrated in FIG. 12. Specifically, a suture 1200 may be looped back through itself at the distal aperture 322 (i.e., lark's head or bear claw knot 1202) and looped back through itself at the drill hole 1102 in the bone plug 1104 (i.e., knot 1204). A hand-tied knot 1206 may be used to close the suture 1200. Alternatively, the suture 1200 may be preformed as a closed loop.

A “hook” could also be used to replace the distal aperture 322 to make use of the lark's head knot. Such a hook may be easier for a metal anchor plug 300. An anchor plug 300 made from a plastic such as polyetheretherketone (PEEK) may not be strong enough to support the tension on the hook.

Where there is tendon fixation without a bone plug and no looping, a suture weave may be used. This may be accomplished a number of ways, both with looped sutures or single-stranded sutures. In a first exemplary aspect, illustrated in FIG. 13, a single-stranded suture may employ a weave (e.g., Krakow as discussed above or similar) along a length of a tendon graft 1300, which will generally result in two free suture ends 1302, 1304 exiting the end of the graft 1300. These suture ends 1302, 1304 may be tied together with a knot 1306 after one is passed through the distal aperture 322. The knot 1306 may be a theoretical point of weakness and/or may impact passage of the graft 1300 into the anchor receptable 400.

Alternatively, the Krakow weave may be done in such a manner that the knot 1306 is tied distally, at a portion 1400 of the graft 1300 further away from the anchor plug 300 as better illustrated in FIG. 14. This approach dramatically reduces stress on the knot 1306. It should be noted that looped sutures may also be used in the same way, allowing for additional fixation options such as the luggage tag/lark's head knot on the anchor plug 300 and/or at either end of the graft 1300.

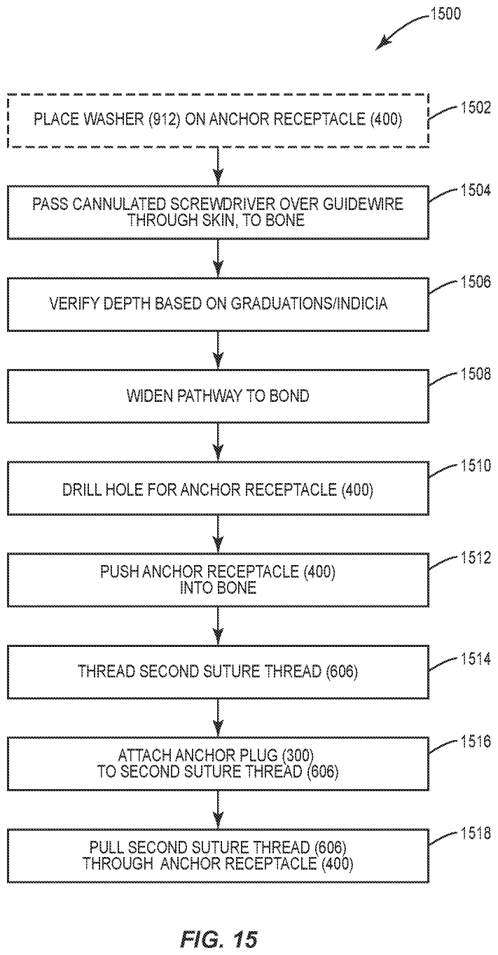

While outlined above, a process 1500 for placing the anchor receptacle 400 is provided again with reference to FIG. 15. The process 1500 starts with the optional placement of a washer 912 over the anchor receptacle 400 (block 1502). Use of the washer 912 may be particularly appropriate if the anchor receptacle 400 is being used on a tibial side of the femur. The cannulated screwdriver may then be passed over the guidewire (beeth pin), through the skin and to the bone (block 1504). The surgeon may also verify the depth by reference to the gradations or indicia on the screwdriver (block 1506).

The pathway is then widened (block 1508). That is, the pathway to the bone through the skin, subcutaneous fat, and muscle may be too small for the anchor receptacle 400 after just using the guidewire/beeth pin. Accordingly, the surgeon may use a scalpel or the like to divide the subcutaneous tissue, fat, and iliotibial band from around the guide wire. A scalpel may be insufficient to get through the muscle without enlarging unnecessarily the entrance incision. Accordingly, femoral or tibial tunnel drill bits can be passed down the guidewire to the bone and spread the muscle tissue out of the way and make sure there are no fibrous bands that might be caught under the flange 410 of the anchor receptacle 400. Instead of the drill bits, a blunt instrument having a diameter approximately equal to the anchor receptacle 400 may be used. This may be a custom instrument or some repurposed instrument. In an exemplary aspect, this instrument may also have graduations or indicia to indicate depth.

A drill may then be used to drill the hole/passage 700 for the anchor receptacle (block 1510). In an exemplary aspect, the drill may be a 6 mm drill and can be passed from outside in or inside out along the beeth pin/guidewire.

With the beeth pin/guidewire still in place, the anchor receptacle 400 may be passed over the beeth pin, pushed by the cannulated screwdriver through the skin and soft tissues into the bone (block 1512).

The second suture thread 606 is then threaded or looped through the end of the beeth pin (block 1514). The anchor plug 300 is then threaded onto the second suture thread 606 (block 1516). The anchor plug 300 will already have been attached to the graft before being attached to the second suture thread 606.

During the steps of blocks 1512, 1514, 1516, the cannulated screwdriver may stay in place, providing counter pressure to keep the anchor receptacle 400 flush on the bone while the beeth pin and the second suture thread 606 are then pulled through the anchor receptacle 400 (block 1518), thereby drawing the anchor plug 300 into the anchor receptacle 400. The screwdriver may then be removed.

Again, note that while repair of an ACL and use of the femur is specifically contemplated, the present disclosure is not so limited and other tendon and ligament repair using other bones may also benefit from the present disclosure. For example, in an exemplary aspect, a hamstring graft may be used. The hamstring graft may be used with sutures attaching the graft to the anchor plug as described above, or, in a further exemplary aspect, the distal aperture 322 may be sized so that the graft may fit therethrough directly. For example, the distal aperture 322 may have a diameter of approximately 6 mm to accommodate such a hamstring graft.

Those skilled in the art will recognize improvements and modifications to the embodiments of the present disclosure. All such improvements and modifications are considered within the scope of the concepts disclosed herein and the claims that follow.

What is claimed is:

1. A fixation assembly comprising:
an anchor plug comprising an elongated body comprising a plurality of tapering conical sections; and
an anchor receptacle comprising an interior passage with interior teeth configured to interengage with the plurality of tapering conical sections of the anchor plug when the anchor plug is inserted into the interior passage.

2. The fixation assembly of claim 1, wherein the anchor receptacle comprises a receptacle body having a longitudinal axis on which lies the interior passage.

3. The fixation assembly of claim 2, wherein the anchor receptacle further comprises a head portion.

4. The fixation assembly of claim 3, wherein the head portion comprises a flange extending outwardly from the receptacle body.

5. The fixation assembly of claim 3, wherein the interior passage passes through the head portion and has a hex-shaped configuration in the head portion.

6. The fixation assembly of claim 2, wherein the receptacle body comprises a distal end.

7. The fixation assembly of claim 6, wherein the distal end comprises an exterior taper.

8. The fixation assembly of claim 6, wherein the distal end comprises an interior taper.

9. The fixation assembly of claim 2, wherein the receptacle body further comprises exterior threads.

10. The fixation assembly of claim 2, wherein the receptacle body comprises resilient clips forming the interior teeth.

11. The fixation assembly of claim 1, wherein the anchor plug comprises a first end and a distal end.

12. The fixation assembly of claim 11, wherein the first end comprises a generally conical tip.

13. The fixation assembly of claim 12, wherein the generally conical tip delimits an aperture therethrough.

14. The fixation assembly of claim 11, wherein the distal end delimits an aperture therethrough.

15. The fixation assembly of claim 11, wherein the distal end comprises an eyelet having an aperture therethrough.

16. The fixation assembly of claim 11, wherein the plurality of tapering conical sections each comprise a lip.

17. The fixation assembly of claim 1, wherein:
the anchor plug comprises a tip and the anchor receptacle comprises a flange; and
the tip extends past the flange but the interior teeth remain interengaged with the plurality of tapering conical sections.

18. The fixation assembly of claim 1, further comprising a second anchor receptacle having a second interior passage sized differently than the interior passage of the anchor receptacle.
